(19) United States
(12) Patent Application Publication
(10) Pub. No.: US 2008/0200844 A1
(43) Pub. Date: Aug. 21, 2008

(54) SURGICAL DEVICE WITH AN IMPACT DETECTOR

(76) Inventors: Manuel Millahn, Munchen (DE); Timo Neubauer, Konigsberg (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/033,389

(22) Filed: Feb. 19, 2008

Related U.S. Application Data
(60) Provisional application No. 60/893,242, filed on Mar. 6, 2007.

(30) Foreign Application Priority Data
Feb. 19, 2007 (EP) 07102641.3

(51) Int. Cl. A61B 5/103

(52) U.S. Cl. 600/595; 600/587

(57) ABSTRACT

A surgical device for performing a medical procedure includes an impact detector operative to provide data indicative of the surgical device being subjected to an impact. A polling device in communication with the impact detector can determine if an impact experienced by the surgical device exceeds a predetermined threshold.

### RELATED APPLICATION DATA

This application claims priority of U.S. Provisional Application No. 60/893,242 filed on Mar. 6, 2007, which is incorporated herein by reference in its entirety.

### FIELD OF THE INVENTION

The present invention relates to surgical devices and, more particularly, to a surgical device that includes an impact detector for detecting the occurrence of impacts suffered by the surgical device. The invention further relates to a polling device for polling information concerning the occurrence of an impact from the surgical device and to a navigation system comprising the polling device.

### BACKGROUND OF THE INVENTION

Surgical devices, such as, for example, instruments, implants or marker means for surgical navigation systems, should have reliable geometric properties, e.g., the devices should be dimensionally stable. An impact, for example due to the surgical device falling on the floor, however, can alter the shape of the surgical device. If a surgical device has suffered an impact, this also can be an indication that the surgical device has become non-sterile and/or has an altered shape, which means that the surgical device can no longer be safely used for surgery.

### SUMMARY OF THE INVENTION

A surgical device in accordance with the invention is configured to provide information that can be used to detect a change in shape (e.g., an undesirable change in shape) in the surgical device. Such change in shape may increase a risk of navigation error, for example, if the damaged device were used in navigated surgery. Examples of such surgical devices include a knife, a biopsy needle, an injection apparatus comprising an injection tip, a drill, etc. The surgical device also can be an implant, such as for example an artificial hip joint, or it can be a trackable marker device (e.g., a reference array) used with surgical navigation systems. In such marker systems, a defined relative position between marker elements (e.g., marker spheres) of the marker device is important, and this relative position can be altered by an impact. Examples of marker devices include reference stars in which marker spheres assume a predetermined position relative to each other due to rigid connecting members. The impact detector can be used to monitor the dimensional stability of the surgical device and/or to determine the risk of a change in shape.

The surgical device can comprise a transponder, e.g., an RFID transponder, microchip transponder, a card transponder or a radio transponder, in which the geometric properties of the surgical device may be communicated to or stored thereon, wherein the geometric properties describe a shape of the surgical device. In the following, the RFID transponder is cited by way of example.

The geometric properties (geometric data) are preferably stored together with the date the geometric properties were measured. If the impact detector of the surgical device indicates an impact of an appropriate intensity (particularly if the impact occurred after the surgical device was calibrated or surveyed), then the geometric properties stored on the transponder are regarded as being invalid.

The impact detector, which can be configured as a passive detector (i.e., the detector has no power supply of its own) serves to detect the occurrence of an impact, wherein the impact detector can alter its state as a result of an impact (e.g., an impact that exceeds a particular threshold). This state represents one example of impact occurrence information that can be retrieved from the transducer.

The impact detector also be designed as an active detector (i.e., the detector has its own power supply) and can comprise an acceleration sensor (also called a crash sensor, shock sensor or impact sensor). The acceleration sensor can provide acceleration data metered over time, which for example can be captured by a microchip or microprocessor and (electrically) stored as data. These sensors also can capture an average value or maximum value of acceleration and emit corresponding signals and/or data. The acceleration data represent examples of impact occurrence information.

The impact sensor of the impact detector is preferably designed such that the action of an impact alters an electrical property of the sensor, which then can be polled to detect the impact. In a particular embodiment, the electrical and/or mechanical property of the impact sensor is permanently altered if the intensity of the impact exceeds a predetermined threshold. The impact sensor, for example, can be configured such that a wire breaks if the intensity of the impact exceeds the predetermined threshold. The break in the wire can cause a significant change in the electrical properties of the sensor (significant increase in resistance and/or high-frequency resonance properties). The electrical property represents an example of impact occurrence information.

The altered resistance, for example, then can be polled by an electric circuit. Alternatively, the altered high-frequency resonance property, which represents the impact occurrence information, can be detected by means of high-frequency waves, similar to or in accordance with a transponder. The impact detector preferably comprises a transponder or is coupled to a transponder such that the altered electrical and/or mechanical properties of the impact sensor are transmitted to the transponder or alter information that can be polled by transponders.

The surgical device is preferably designed such that the impact detector can be removed and/or exchanged and/or reset to its original state (e.g., the state prior to impact). This is particularly advantageous when the impact detector permanently alters its (electrical) state due to a (sufficiently intense) impact, wherein an impact is to be regarded as any type of occurrence that is a mechanical burden that may damage or even destroy the surgical device. Maintenance then can be performed on the surgical device such that the impact detector can be made “active” again by replacing or resetting the sensor or sensor element (e.g., analogous to replacing a fuse or resetting a circuit breaker in an electrical circuit). During maintenance, the geometric properties of the surgical device can be resurveyed (i.e., the surgical device can be recalibrated). The newly measured data, for example, then can be stored in a transponder, in particular an RFID transponder, situated within the surgical device. In this respect, reference is also made to European patent application No. 06 019 346.3 (U.S. patent application No. 60/826,973), which describes a measuring device for measuring geometric properties of a medical treatment device, wherein an RFID writer writes the geometric properties (geometric data) of the medical treatment device in an RFID transponder situated in the medical treatment device. The apparatus recalibrated in this way and comprising a replaced or re-activated impact detector then can be used again. The surgical device in accordance with the present invention can comprise the features of the medical treatment device of European patent application No. 06 019 346.3 or U.S. patent application No. 60/826,973, the contents of which are hereby incorporated by reference in their entireties.

As stated above, the impact detector can be directly polled, in particular electrically, by a polling apparatus. To this end, electrodes, for example, can be provided on the surgical device, and a polling apparatus can be connected to these electrodes so as to enable measurement of the resistance or capacitance of the impact detector. Preferably, however, polling is contact-free via an electrical alternating field, for example, in particular a high-frequency field that detects an altered resonance frequency of the impact detector.

In accordance with another embodiment, the surgical device comprises a transponder, in particular an RFID transponder, which for example can (but need not) store geometric data concerning the surgical device. The transponder can be used to store the information ascertained by an impact detector concerning the occurrence of an impact and/or to transmit the information to a transponder reader, such as an RFID reader. To this end, the transponder is preferably electrically coupled to the impact detector. The impact detector, for example, can transmit data to the transponder, or the transponder can poll said data from the impact detector, wherein the data reflects the occurrence of an impact. These data then can be stored in memory by the transponder and retrieved therefrom as necessary by a transponder reader.

Even if the impact detector is designed as a passive detector, the value of a memory cell, in particular the binary value, can be altered, for example by altering a resistance value of the impact detector if an impact is sufficiently intense. The altered memory contents of the memory cell are then in turn read by a transponder reader, such as an RFID reader, making it possible to determine whether or not the surgical device has been exposed to an impact.

A polling device in accordance with the invention is designed to poll the surgical device to read impact occurrence information and/or geometric data stored in a transponder, such as an RFID transponder, wherein the geometric data represent the geometric properties of the surgical device. The polling device preferably also comprises a comparing unit which compares impact tolerance information with the polled impact occurrence information. The impact occurrence information, for example, represents acceleration data recorded by the impact detector or reflects the state of the impact detector (e.g., if it is designed as a binary detector that only conveys whether or not an impact has occurred). The polled impact occurrence information is preferably compared by the comparing unit with impact tolerance information. This applies in particular when the impact occurrence information includes information concerning the intensity of the impact and/or acceleration that the surgical device has suffered. Such impact occurrence information can be compared with impact tolerance information in order to determine whether the intensity of the impact was above a threshold. If the intensity of the impact was above the threshold, then a risk of damage to the surgical device or of a change in the shape of the surgical device can be assumed. In particular, it may be assumed that the geometric data, which in one embodiment are additionally output from the transponder (e.g., via an RFID transponder) of the surgical device, are no longer reliable.

The polling device is preferably configured such that, in addition to the impact occurrence information, also reads identification data from the surgical device. The identification data, for example, can be stored in a transponder, in particular an RFID transponder, provided in the surgical device. On the basis of the identification data read from the surgical device, a database can then be polled in which different impact tolerance information is respectively assigned to the identification data. The impact tolerance information, for example, represents the intensity of an impact value at which the risk of a change in shape is not yet to be anticipated or beyond which such a risk is to be anticipated. The impact tolerance information is preferably assigned to the individual types of surgical devices by means of the identification data. Different types of surgical devices can have different impact tolerances. In this way, it is then possible to individually determine, depending on the type of surgical device, whether or not there is a risk of deformation. The polling device preferably comprises the aforesaid database. The polling device preferably comprises a warning unit that warns a user if the result of the comparison made by the comparing unit indicates a risk of deformation, e.g., indicates that a tolerance has been exceeded. In particular if the transponder additionally stores the geometric data of the surgical device, it is possible, if there is such a risk, to block the geometric data from being read from or relayed, or to only enable the geometric data to be relayed in combination with a warning indication.

A system in accordance with the present invention can include the aforementioned surgical device, polling device, and/or a navigation system, in particular for performing IGS (image-guided surgery). Preferably, the polling device is included in the navigation system. If the polling device determines that a tolerance has been exceeded or that there is a risk of deformation, the navigation system preferably outputs a warning signal and/or blocks navigation of the surgical device for which a risk of deformation has been determined. In this way, it is possible to prevent deformed surgical devices from being used in medical surgery. In particular, it is possible to prevent geometric data of the surgical devices which are no longer valid due to an impact, but which are still stored in a transponder of the surgical device and are read by the polling device of the navigation system, from being assigned by the navigation system. This avoids faulty or risky geometric data being used in navigating the surgical device. In particular, it can cause the geometric properties of the surgical device to be newly surveyed and stored in the transponder, in particular an RFID transponder, before the surgical device is used again.

### BRIEF DESCRIPTION OF THE DRAWINGS

The forgoing and other features of the invention are hereinafter discussed with reference to the drawing.

FIG. 1 schematically shows an exemplary surgical device and polling device in accordance with the invention.

FIGS. 2a and 2b schematically show an exemplary non-reusable impact sensor that can be used with the surgical device of FIG. 1.

### DETAILED DESCRIPTION

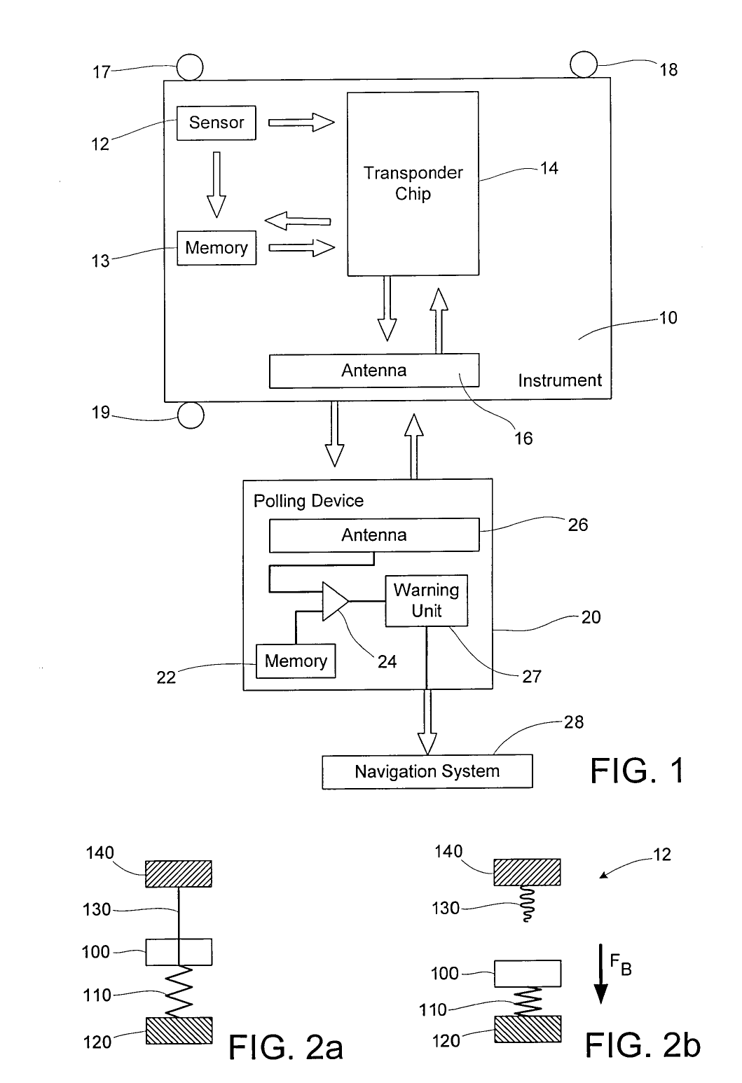

FIG. 1 schematically shows the design of an exemplary surgical device 10 and a polling device 20 in accordance with the invention. A trackable marker device (also referred to as reference units) such as, for example, passive or active marker spheres 17, 18 and 19, are attached to the surgical device 10 and detectable by a detection device (e.g., camera of a navigation system). In the example shown, the surgical device is a medical instrument (e.g., a pointer). An impact sensor 12 is coupled to the device 10 and transmits (e.g., electrically) data concerning the occurrence of an impact to a transponder chip 14 (e.g., an RFID tag). Impact information detected by the impact sensor 12 as well as identification information that uniquely identifies the surgical device 10 and/or geometric data of the surgical device 10 may be stored in memory for later use. The memory may be a separate memory 13, for example, or the memory may be formed within the sensor 12 itself. Alternatively, the memory may be formed within the transponder chip 14.

The transponder chip 14 is coupled to antenna 16 for receiving and transmitting signals (e.g., the transponder can output signals to the antenna 16 for wireless transmission of the signals). The antenna 16 interacts with a high-frequency electromagnetic alternating field emitted by an antenna 26 of a transponder reader, in particular an RFID reader 20. Using this interaction, signals can be transmitted to the instrument 10, more specifically to the transponder chip 14, and signals from the transponder chip 14 can be received by the transponder reader, in particular the RFID reader 20. The transponder reader, for example, can relay the received data to a navigation system 28 or can be part of such a navigation system. The transponder reader 20 represents an example of a polling device in accordance with the invention.

The polling device 20 may include a memory 22 for storing data therein. This data can include, for example, identification information of the surgical device 10 (e.g., a code that identifies a characteristic of the device or identifies the device itself), tolerance data (e.g., maximum impact data), etc. that may be used by the polling device 20 (or other device) to determine if the surgical device 10 has undergone an impact exceeding a predetermined threshold. The polling device 20 also includes a comparator unit 24, wherein the comparator unit receives impact information from the surgical device 10 via antenna 16 and antenna 26, as well as tolerance information from memory 22. By comparing the tolerance information and the impact information, a determination can be made whether or not the surgical device 10 has undergone an impact that exceeds a predetermined threshold. If the device has undergone an impact that exceeds the predetermined threshold, then this information can be communicated via warning unit 27, which is operatively coupled to the comparator unit 26.

The sensor 12, for example, can be an impact sensor or an accelerometer. Examples of impact sensors and their applications are for example described in the following documents, the contents of which are hereby incorporated by reference in their entirety.
EP 1 258 896
JP 2004-093274
JP 2002-350459
JP 2001-289873
U.S. Pat. No. 5,970,794
GB 2,424,916
U.S. Pat. No. 5,731,957
US 2001-002451
DE 103 34 700
WO 2004049280
WO 03085617
WO 2005117691
WO 9924093
WO 0250554
JP 2004-093274 and the corresponding English abstract disclose an acceleration sensor which, with no power supply, detects an impact event and alters its state as a result of the impact event. More specifically, the capacitance of the device is altered or, if a piezoelectric element is used, the resistance of the piezoelectric element is altered. In accordance with the invention described in JP 2004-093274, an inert mass part is held by an elastic pivoting mechanism, which can assume two positions. If the device is exposed to an acceleration exceeding a predetermined threshold, the inert mass part can change from one position to the other, which in particular alters the electrical capacitance or, if piezoelectric elements are used along the elastic hinge, alters the resistance of the piezoelectric elements. The acceleration sensor described in JP 2004-093274 can be configured to be reusable if the inert mass part is returned to its initial position again. The transponder chip 14, for example, also can store the geometric data of the instrument 14 and/or identification information that identifies the instrument 14. These data and/or this information also can be output by the transponder reader, in particular the RFID reader 20.

FIGS. 2a and 2b show the principle of operation of a non-reusable impact sensor 12. An inert mass part 100 is connected to an electrode 120 via a spring 110. In FIG. 2a, the electrode 120 is connected via the spring 110 and the inert mass part 100 to a wire 130, and a tension is placed on the wire. The wire 130 is in turn electrically connected to the electrode 140, such that a current can flow between the electrode 140 and the electrode 120. If an acceleration force FB occurs, as shown in FIG. 2b, then the inert mass part 100 is moved towards the electrode 120, such that the elastic element 110 is compressed. This movement breaks the wire 130, which then for example assumes its pre-set, relaxed state, which is shorter than its state shown in FIG. 2a. The electrical connection between the electrode 140 and the electrode 120 is thus interrupted by the occurrence of an impact that exerts the acceleration force FB on the inert mass part 100.

The impact sensor in accordance with FIGS. 2a and 2b thus alters its resistance due to the occurrence of an impact. This change in resistance can in turn be polled by a transponder chip 14 or also in general by an electromagnetic alternating field. In the latter case, antennae, for example, can be attached to the electrodes 120 and 140, wherein the resonance property of the antenna can be altered by the wire 130 breaking. The altered resonance property can be detected by a polling device that emits high-frequency waves. Detecting the altered resonance property then indicates the occurrence of an impact which the instrument has suffered.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A surgical device for performing a medical procedure, comprising an impact detector operative to provide data indicative of an impact subjected on the surgical device.

2. The surgical device according to claim 1, wherein the surgical device is an instrument, an implant or a trackable marker device for surgical navigation systems.

3. The surgical device according to claim 1, wherein the impact detector retrievably stores impact occurrence information.

4. The surgical device according to claim 1, wherein the impact detector is a passive impact detector.

5. The surgical device according to claim 1, wherein the impact detector is configured to permanently alter an electrical property of the impact detector as a result of an impact exceeding a predetermined threshold.

6. The surgical device according to claim 1, wherein the impact detector comprises an acceleration sensor.

7. The surgical device according to claim 1, comprising a transponder operatively coupled to the impact detector, said transponder operatively configured to poll data generated by the impact detector.

8. The surgical device according to claim 7, wherein the transponder and the impact detector are formed as an integral unit.

9. The surgical device according to claim 1, comprising a memory for storing data, wherein data derived by the impact detector is stored in said memory.

10. The surgical device according to claim 1, comprising a memory for storing acceleration values detected by the impact detector.

11. The surgical device according to claim 1, comprising a transponder operatively coupled to the impact detector, wherein the transponder is configured to store impact occurrence information from the impact detector and/or to communicate the impact occurrence information to a reader.

12. The surgical device according to claim 1, comprising an RFID transponder operative to store geometric data and/or identification data corresponding to the surgical device.

13. A system for monitoring a risk of damage to a device, comprising:
a surgical device for performing a medical procedure, said surgical device including an impact detector operative to provide data indicative of an impact subjected on the surgical device; and
a polling device operative to poll the surgical device to obtain impact occurrence information detected by the impact detector.

14. The system according to claim 13, wherein the polling device comprises a comparing unit operative to compare impact tolerance information with the polled impact occurrence information.

15. The system according to claim 14, comprising a warning unit operative to output a warning signal when the result of the comparison indicates that a tolerance has been exceeded.

16. The system according to claim 13, wherein the surgical device includes identification information stored thereon, said identification information indicative of a characteristic of said surgical device, and wherein the polling device includes a plurality of impact tolerance information and corresponding identification information stored in memory, said polling device configured to poll the identification information from the surgical device, and to retrieve impact tolerance information from memory corresponding to the polled identification information, and to compare the retrieved impact tolerance information with the polled impact occurrence information.

17. The system according to claim 13, wherein the polling device is configured to obtain geometric data from a transponder of the surgical device.

18. The system according to claim 13, comprising a navigation system communicatively coupled to said polling device, wherein when the result of the comparison indicates that a tolerance has been exceeded, said navigation system configured to i) output a warning signal and/or ii) disable navigation of the surgical device.

19. The system according to claim 18, wherein when the result of the comparison indicates that a tolerance has been exceeded, geometric data obtained from the surgical device are not used or are only used with a warning indication.
